(19) United States
(12) Patent Application Publication
(10) Pub. No.: US 2023/0372350 A1
(43) Pub. Date: Nov. 23, 2023

(54) ASTROCYTIC TRPA1 CHANNEL INHIBITION AS A NOVEL NEUROPROTECTIVE THERAPEUTIC TARGET IN THE PRODROMAL PHASES OF ALZHEIMER'S DISEASE

(71) Applicants: UNIVERSITE GRENOBLE ALPES, Saint Martin D'Heres (FR); INSTITUT NATIONAL DE LA SANTE ET DE LA RECHERCHE MEDICALE, Paris (FR)

(72) Inventors: Mireille ALBRIEUX, La Tronche (FR); Adrien PAUMIER, La Tronche (FR); Anthony BOSSON, La Tronche (FR); Alain BUISSON, La Tronche (FR); Sylvie BOISSEAU, La Tronche (FR)

(73) Assignees: UNIVERSITE GRENOBLE ALPES, Saint Martin D'Heres (FR); INSTITUT NATIONAL DE LA SANTE ET DE LA RECHERCHE MEDICALE, Paris (FR)

(21) Appl. No.: 18/027,190

(22) PCT Filed: Sep. 17, 2021
(86) PCT No.: PCT/IB2021/058487

§ 371 Date: Mar. 20, 2023

(30) Foreign Application Priority Data
Sep. 18, 2020 (FR) 20/09509

(51) Int. Cl. A61K 31/522; A61P 25/28

(52) U.S. Cl. CPC A61K 31/522; A61P 25/28

(57) ABSTRACT

A TRPA1 calcium channel inhibitor for use as a neuroprotectant in the treatment and/or prevention of the early stages of Alzheimer's disease and as an anti-inflammatory in the treatment and/or prevention of neuroinflammatory processes in Alzheimer's disease. In particular, the inhibitor HC030331 of the TRPA1 calcium channel for its use as a neuroprotectant in the treatment and/or prevention of the early stages of Alzheimer's disease and as an anti-inflammatory in the treatment and/or the prevention of the neuroinflammatory processes of Alzheimer's disease. A pharmaceutical composition comprising at least the inhibitor of the invention and at least one pharmaceutically acceptable excipient.

### BACKGROUND OF THE INVENTION

The present invention concerns the field of the treatment and prevention of neurodegenerative diseases, in particular Alzheimer's disease, and relates in particular to the neuroprotection against Alzheimer's disease.

Alzheimer's disease is the main cause of dementia in humans and is accompanied by a severe loss of the main cognitive and memory functions. There is currently no cure for the disease and the failure of recent therapeutic strategies has highlighted the urgent need for effective therapeutic targets.

This pathology begins long before the appearance of memory disorders and this long asymptomatic phase is the one that future therapies will have to target in order to prevent the mechanisms of neurodegeneration. Recent studies have highlighted prodromal warning symptoms characterized by abnormally high neuronal activity in specific circuits within the hippocampus in humans and animal models of Alzheimer's disease. This neural hyperactivity has been associated with memory deficits that do not significantly alter the daily activity (Haberman et al., Neurotherapeutics 2017). It is at this early phase that we are interested in this patent application.

At the level of the central nervous system, astrocytes can no longer be considered solely as support cells and it is now accepted that they influence their environment and functionally modulate neighboring neuronal cells. Notably, they may be important contributors to the progression of neurodegenerative diseases.

Their role in Alzheimer's disease has mainly been observed in the advanced stages of the pathology where the astrocyte inflammatory phenotype and astrogliosis have been associated with the formation of senile plaques (Osborn et al., Prog. Neurobiol. 2016; Kuchibhotla et al., Science 2009).

Surprisingly, the involvement and role of astrocytes in the early stages of Alzheimer's disease remain largely unexplored. However, apart from astrogliosis and inflammation, changes in some astrocyte functions, such as reduced clearance of toxic substances, imbalance of local neuronal activity, glutamatergic dyshomeostasis, could amplify or even induce a deleterious synaptic toxicity for neurons.

The inventors have highlighted that these cells were involved in the establishment of early neuronal hyperactivity of hippocampal networks in a murine model of Alzheimer's disease. They have identified a channel expressed by these astrocyte cells, the TRPA1 channel, which plays a crucial role in the establishment of this hyperactivity.

The TRPA1 channel was initially highlighted in a subpopulation of nociceptive neurons from the sensory ganglia of the dorsal root of the spinal cord, the trigeminal nucleus and the nodosum (Lee et al., J. Chem. Neuroanat. 2012). It is particularly involved in the nociceptive transmission of acute inflammatory pain. In the trigeminal nucleus, an expression of TRPA1 has been shown in some nociceptive neurons but also in the fine extensions of astrocytes. This astrocyte expression increases in the event of inflammation (Lee S M et al., J. Chem. Neuroanat. 2012). At the level of the central nervous system, the expression of the TRPA1 channel is poorly described. However, astrocyte-specific expression has been reported in rat and mouse hippocampus. In rats, this astrocyte TRPA1 channel has been involved in the regulation of the level of extracellular GABA and therefore of the inhibitory transmission of GABAergic interneurons in the hippocampus (Shigetomi et al., Nat. Neurosci. 2012). In mice, the same research team has highlighted an involvement of astrocyte TRPA1 in the phenomenon of long-term potentiation via a release of astrocyte D-serine (Shigetomi et al., J. Neurosci. 2013).

In the pathological context of Alzheimer's disease, an anti-inflammatory role of the TRPA1 channel has been described in the late stages of the disease (8 months in the mouse model). This anti-inflammatory role has been related to a release of astrocyte pro-inflammatory cytokines (Lee K I et al., J. Neuroinflammation 2016).

HC030031 is a TRPA1 specific inhibitor (CI₅₀˜6.2 μM) whose binding site has been identified in the intracellular portion “linker” of the channel, between the TM4 and TM5 transmembrane domains (McNamara et al., Proc. Natl. Acad. Sci. U.S.A. 2007; Gupta et al., Sci. Rep. 2016). The oral administration of HC030031 (100 mg/kg) showed a significant analgesic effect in models of neuropathic and inflammatory pain in rats (Eid et al., Mol. Pain. 2008). Subcutaneous (100 μg/10 μl) or intragastric (300 mg/kg) administration of HC030031 also abolished trigeminal neuropathy-like pain behaviors in mice (Trevisan et al., Brain 2016). These studies validate the use of this pharmacological substance in studies in rodents.

The inventors of the present invention have focused their attention on the early stages of Alzheimer's disease, in particular the so-called the “prodromal stage” which is increasingly studied in humans. This heralding stage precedes the appearance of the first symptoms by several years.

During the prodromal stage of Alzheimer's disease, the subject presents mild memory difficulties, changes in his behavior or his mood. These difficulties are associated with hyperactivity of the hippocampus network (seat of memory) detected by medical imaging and which would prevent the correct coding of new memories (Haberman et al., Neurotherapeutics 2017). Nevertheless, at this stage, performance in neuropsychological tests may be preserved and may not allow the detection of a warning sign of the disease. Neurobiologically, biomarkers such as amyloid β peptide are already present.

It is therefore on the events preceding the appearance of clinical manifestations, in particular memory disorders, that the subject of the present invention will relate.

### BRIEF DESCRIPTION OF THE INVENTION

The inventors have highlighted that the TRPA1 channel is responsible for early astrocyte calcium hyperactivity in the CA1 region of the hippocampus in a murine model of Alzheimer's disease (APP/PS1-21 animals aged 1 month).

This astrocyte hyperactivity is inhibited by the application of a TRPA1 channel inhibitor, HC030031, at 40 μM on acute hippocampal slices (in vitro). The inventors have also shown that inhibition of this TRPA1 channel restores the hippocampal neuronal hyperactivity characterizing the early phases of Alzheimer's disease and announcing memory symptoms.

Thus, this TRPA1 channel has a potential involvement in the establishment of synaptotoxicity and neuronal dysfunctions long before the appearance of the first senile plaques (>3 months in mice), the inflammatory processes (>4 months in mice) or the memory and cognitive disorders (>8 months in mice).

The results observed by the inventors make it possible to consider the importance of TRPA1 as a therapeutic target in the early stages of Alzheimer's disease. Indeed, in vivo, the TRPA1 specific inhibitor, HC030031, administered to animals restores the neuronal activity of the hippocampus to its basal level at the prodromal stage in mice models of Alzheimer's disease.

Pharmacological inhibition by a TRPA1 channel inhibitor such as HC030031 is considered to prevent the onset of neuronal toxicity of amyloid β peptide and is thus considered to play a neuroprotective role in the early phases of Alzheimer's disease.

Thus, chronic treatment with the TRPA1 channel inhibitor which crosses the blood-brain barrier would prevent the establishment of deleterious neuronal effects and therefore the occurrence of memory disorders and cognitive decline which accompany the more advanced stages of Alzheimer's disease.

This neuroprotective effect of the TRPA1 channel inhibitor such as HC030031 and the use thereof in the prevention and/or treatment of the early stages of Alzheimer's disease is particularly innovative given the fact that the prodromal stages remain understudied.

The inventors have also highlighted that inhibition of the TRPA1 channel restores some phenomena of inflammation of the central nervous system responsible for pathological flare-up at a slightly later stage.

Furthermore, the TRPA1 channel is a target of particular interest since it seems to have little physiological impact on astrocyte function while being brought into play very early in the event of aggression, in particular that due to toxicity characterized by the presence of amyloid β peptide. Its specific inhibition by the inhibitor HC030031 or any other appropriate inhibitor would make it possible to block the neuronal consequences (dysfunction then neuronal death) of the aggression with potentially few adverse effects, which constitutes a considerable advantage from a clinical point of view.

Indeed, the data obtained by the inventors showed that chronic blockade of the TRPA1 channel in healthy WT (wild-type) mice had no effect on astrocyte activity, neuronal function, structure synapses and memory performance. This reinforces the relevance of the TRPA1 channel as a therapeutic target.

The observation that the preclinical hippocampal hyperactivity is an early neural dysfunction implicated in disease progression represents a major conceptual shift in understanding Alzheimer's disease, particularly as it appears to be remediable if acted upon at this early stage.

### DETAILED DESCRIPTION OF THE INVENTION

The present invention concerns a TRPA1 calcium channel inhibitor for use as a neuroprotectant in the treatment and/or prevention of the early stages of Alzheimer's disease and as an anti-inflammatory in the treatment and/or prevention of neuroinflammatory processes in Alzheimer's disease.

Herein, the term “early stages” of Alzheimer's disease means the prodromal phase, i.e. the announcing stage which precedes, in humans, by several years the appearance of the first symptoms. During the prodromal stage of Alzheimer's disease, the subject presents mild memory difficulties, changes in his behavior or his mood. These difficulties are associated with a hyperactivity of the hippocampus network (seat of memory) detected by medical imaging. Nevertheless, at this stage, performance in neuropsychological tests may be preserved and may not allow the detection of a warning sign of the disease. Neurobiologically, biomarkers such as amyloid β peptide are already present.

A distinction is made between genetic familial forms of Alzheimer's disease and sporadic forms. Familial forms are hereditary and represent approximately 1% of Alzheimer's disease cases and are manifested by an early form of the pathology, which is triggered around the age of 40 in these subjects. The other forms, sporadic, are triggered later. The average age at which the disease appears is around 70-80 years. A growing proportion of the world's population is affected by this sporadic form.

The highlighting of the prodromal stage in these subjects can be performed:
by means of genetic tests which make it possible to identify subjects carrying mutations (in particular PSEN1, PSEN2 and APP) for family forms (these tests are offered to families identified with the establishment of genetic advice);
by means of MRI-type neuroimaging highlighting neuronal hyperactivity in the hippocampus, as well as by CT, PET positron emission tomography or EEG electroencephalography, and also of the detection of blood biomarkers (for example, the isoform Aβ-42, total tau protein t-tau, p-tau, a plurality of metabolites including choline, L-carnitine, creatinine, acylcarnitines, lysophospholipids, sphingomyelins, C22:0 and C24:0 ceramides, amino acids; see Wang et al., July 2021, Frontiers in Pharmacology, Volume 12; Henriques de Aquino, August 2021, Frontiers in Neurology, Volume 12) which make it possible to detect the disease very early, before the appearance of symptoms for sporadic forms. These diagnostic methods are in full development and the identification of blood biomarkers will probably allow an early diagnosis on a large scale in the short/medium run.

Neuroprotection by means of the inhibitor of the present invention can be ensured very early before the appearance of the first symptoms of Alzheimer's disease, during the prodromal stage. This stage has been extensively described and is known as “mild cognitive impairment” (MCI). Alzheimer's disease assessment scales have been developed (of the type Alzheimer's Disease Assessment Scale-Cognitive (ADAS-Cog), Clinical Dementia Rating Scale sum of boxes (CDR-SB)), which correlate via a notation with impairments identified in the subject with the disease stage. Acting from the prodromal stage as proposed by the invention is essential in the fight against this disease, which, once the symptoms have appeared, is accompanied by irreversible neurodegeneration.

The “neuroinflammatory processes” correspond to astrogliosis and microgliosis which occur in the more advanced stages of Alzheimer's disease, manifested by a modification of glial functions which results in particular in the secretion of pro-inflammatory molecules. These deleterious neuroinflammatory processes are associated with the formation of amyloid plaques and participate in the pathogenesis of Alzheimer's disease.

“Astrocytes” are glial cells of the central nervous system and have an important role in supporting and protecting neurons. Astrocytes are able to provide a form of communication, based on intracellular waves of calcium or “calcium events” which are transmitted to neighboring astrocytes. Under physiological conditions (healthy brain), astrocytes have a basal spontaneous calcium activity which contributes to maintaining the structural and functional integrity of synapses. In the early stages of Alzheimer's disease, the presence of soluble oligomeric forms of amyloid beta peptide (Aβ) disrupts astrocyte function. Through an activating effect of the TRPA1 calcium channel, the astrocyte becomes hyperactive, which has the consequence of inducing a hyperactivity of neighboring neurons. These combined phenomena eventually lead to the phenomenon of synaptic toxicity which results in neuronal hyperactivity, collapse of dendritic spines, astrocyte stripping of synapses and a dysfunction of neuronal communication (late pre-symptomatic stage). This ultimately leads to a neuronal death, a prelude to memory disorders and dementia (symptomatic stage of Alzheimer's disease).

The inhibitor of the present invention can be selected from the group consisting of HC030031, Chembridge-5861528, A-967079, AP-18, GRC-17536, CB-625, ODM-108, GSK205, GDC-0334 and their derivatives. Other inhibitors known mainly for their analgesic properties which have been presented in PCT patent applications WO2015103060 in the name of Algomedix, WO2015155306, WO2017060488, WO2017064058 in the name of Almirall, WO2016028325, WO2017177200 in the name of Duke University/The Regents of the University of California, WO2015115507, WO2017018495 and WO2017135462 in the name of EA Pharma, WO2019152465 in the name of Eli Lilly and Company, WO2018109155 in the name of Galderma, WO2014049047, WO2015052264, WO2016128529, WO2018015410, WO2018015411, WO2018029288, WO2018096159, WO2018162607 and WO2019182925 in the name of Genetech, Inc., WO2015056094 and WO2016042501 in the name of Glenmark Pharmaceuticals, WO2015164643 and WO2016044792 in the name of Hydra Biosciences, WO2015002095 in the name of Kao Corporation, WO2018180460 in the name of Mandom Corporation, WO2015144976 and WO2015144977 in the name of Orion Corporation, WO2016067143 in the name of Pfizer, Inc. may also be used.

Salts and derivatives of the compounds mentioned hereinabove, and their pharmaceutically acceptable forms are also contemplated in the present invention.

In a preferred embodiment, the inhibitor of the present invention is HC030331 of the following formula:

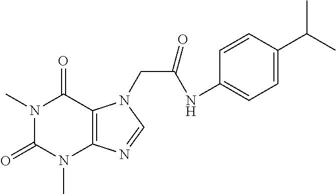 [SMILES: CC(C)c1ccc(NC(=O)Cn2cnc3c2c(=O)n(C)c(=O)n3C)cc1]

HC030331 is a TRPA1 channel specific inhibitor discovered by Hydra Bioscience in 2007.

In one embodiment, the TRPA1 calcium channel inhibitor may be intended to be administered to a subject in need thereof from the prodromal phase of Alzheimer's disease characterized by neuronal hyperactivity in the hippocampus detectable by brain imaging and mild memory disorders or associated with hereditary genetic forms of the disease in identified families at risk. Studies are ongoing on the identification of early biomarkers in humans that can then be used to better identify this prodromal phase as a replacement or in addition to brain imaging.

Thus, in persons at risk (familial Alzheimer's disease) or in a context where diagnostic methods are evolving (development of early biomarkers) or if diagnosis by brain imaging is carried out (of the functional Magnetic Resonance Imaging type (fMRI, Habermann et al., Neurotherapeutics 2017) and highlights a neuronal hyperactivity in the hippocampus, it would become possible to administer the inhibitor early and stop the irreversible neurodegeneration processes.

With regard to early biomarkers, many studies are underway to identify biomarkers detectable in the blood, which could facilitate and extend diagnosis (for review, see Scheltens et al., The Lancet, 2016). At that time, the use of the inhibitor will be possible for prevention in a large population.

By “hippocampus” it should be understood the structure of the telencephalon, which plays a key role in memory and space navigation. The hippocampus is one of the first structures affected in Alzheimer's disease, which explains the memory problems and disorientation that mark the emergence of this neurodegenerative disease. A hyperactivity of this structure prevents the correct coding of the memorization and progressively destroys the existing connections. It then generates a deleterious vicious circle that leads to irreversible degeneration, hence the interest of targeting this early phase.

In a particular embodiment, the inhibitor may be intended to be administered to a subject in need thereof in a pharmaceutically effective amount.

The term “subject” refers to an animal or human subject of both genders.

The expression “in need thereof” refers herein to a subject with indications of the prodromal stage of Alzheimer's disease. This can be manifested in the form of mild memory difficulties, changes in his behavior or his mood. These difficulties are associated with a hyperactivity of the hippocampus network (seat of memory) detected by medical imaging or using blood biomarkers, or are associated with hereditary genetic forms of the disease in identified families at risk.

By “pharmaceutically effective amount”, it should be understood the amount which is sufficient to perform the treatment or prevention when administered to a subject in need of such treatment or prevention. The therapeutically effective amount depends on the subject, the stage of the disease to be treated and/or prevented and the mode of administration, and can be determined by routine operations by those skilled in the art. It thus may vary with the age and sex of the subject.

Furthermore, the specific therapeutically effective dose for any patient will depend on a variety of factors including the disorder being treated and the severity of the disorder; the activity of the used specific compound; the used specific composition, the age, body mass, general state of health, sex and diet of the patient; the duration of administration, route of administration, and rate of excretion of the used specific compound; the duration of treatment; drugs used in combination or simultaneously with the used specific compound; and similar factors well known in the medical art. For example, it is well within the skill of those skilled in the art to start doses of the compound at rates lower than those required to obtain the desired therapeutic effect and to gradually increase the dosage until the effect desired is obtained.

As an example, in animals, the inhibitor can be administered at a concentration of the order of 100 mg/kg of body weight by oral administration or of 5 mg/kg of body weight by intraperitoneal or subcutaneous route of administration. The determination of the concentration and the mode of administration of the inhibitor can be carried out by routine operations by those skilled in the art.

In a particular embodiment, the inhibitor is intended to be administered by any one of the routes of administration selected from the group consisting of the oral route, the intravenous route, the intra-arterial route, the intradermal route, the intraperitoneal route, the intracardiac route, the intracerebroventricular route, the transdermal route, the topical route, the subcutaneous route, the nasal route or the pulmonary route.

Thus, the mode of administration can be by injection or by gradual infusion. The injection can be intravenous, intraperitoneal, intramuscular, subcutaneous or transdermal.

Preferably, the inhibitor is intended to be administered orally or intravenously.

Examples of forms suitable for oral administration include, but are not limited to, tablets, orodispersible tablets, effervescent tablets, powders, granules, pills (comprising sweetened pills), sugar-coated pills, capsules (comprising soft gelatin capsules), syrups, liquids, gels or other solutions, suspensions, slurries, liposomal and similar forms.

Examples of forms suitable for injection comprise, but are not limited to, solutions, such as, for example, sterile aqueous solutions, dispersions, emulsions, suspensions, solid forms suitable for use in preparing solutions or suspensions by adding a liquid before use, for example, a powder, liposomal or similar forms.

An inhibitor such as HC030031, thanks to its small size and lipophilicity, can cross the blood-brain barrier and can therefore be delivered to the brain without difficulty.

The invention concerns a pharmaceutical composition for use in the treatment and/or prevention of the early stages of Alzheimer's disease and in the treatment and/or prevention of the neuroinflammatory processes of Alzheimer's disease, comprising at least the inhibitor of the invention and at least one pharmaceutically acceptable excipient.

By “pharmaceutically acceptable excipient”, it should be understood a non-toxic material which does not interfere with the effectiveness of the biological activity of the active principles of the composition and which is compatible with a biological system such as a cell, a cell culture, a tissue or an organism. The characteristics of the excipient will depend on the mode of administration. This comprises any solvent, dispersing medium, coatings, antibacterial and antifungal agents, isotonic agents and absorption retarding agents, and the like. A pharmaceutically acceptable carrier or excipient refers to a non-toxic solid, semi-solid or liquid filler, a diluent, an encapsulating material or an accessory formulation of any type.

In a particular embodiment, the composition is intended to be administered to a subject in need thereof in a pharmaceutically effective amount.

In another particular embodiment, the composition is intended to be administered to a subject in need thereof by any one of the routes of administration selected from the group consisting of the oral route, the intravenous route, the intra-arterial route, the intradermal route, the intraperitoneal route, the intracardiac route, the intracerebroventricular route, the transdermal route, the topical route, the subcutaneous route, the nasal route or the pulmonary route.

Preferably, the composition can be administered orally or intravenously.

In a particular embodiment, the composition is intended to be administered to a subject in need thereof from the prodromal phase of Alzheimer's disease characterized by neuronal hyperactivity in the hippocampus detectable by brain imaging and mild memory disorders or associated with hereditary genetic forms of the disease in identified families at risk.

The present invention also concerns a method for treating and/or preventing the early stages of Alzheimer's disease and a method for treating and/or preventing the neuroinflammatory processes of Alzheimer's disease using the inhibitor or the composition described above.

For this purpose, the inhibitor or the composition of the invention is administered to a subject in need thereof from the prodromal phase of Alzheimer's disease characterized by neuronal hyperactivity in the hippocampus detectable by brain imaging or using blood biomarkers and mild memory disorders, or associated with hereditary genetic forms of the disease in identified families at risk.

To better illustrate the object of the present invention, a description will now be given hereinbelow, by way of illustration and not as a limitation, of the examples hereinafter in conjunction with the appended drawings.

In these drawings:

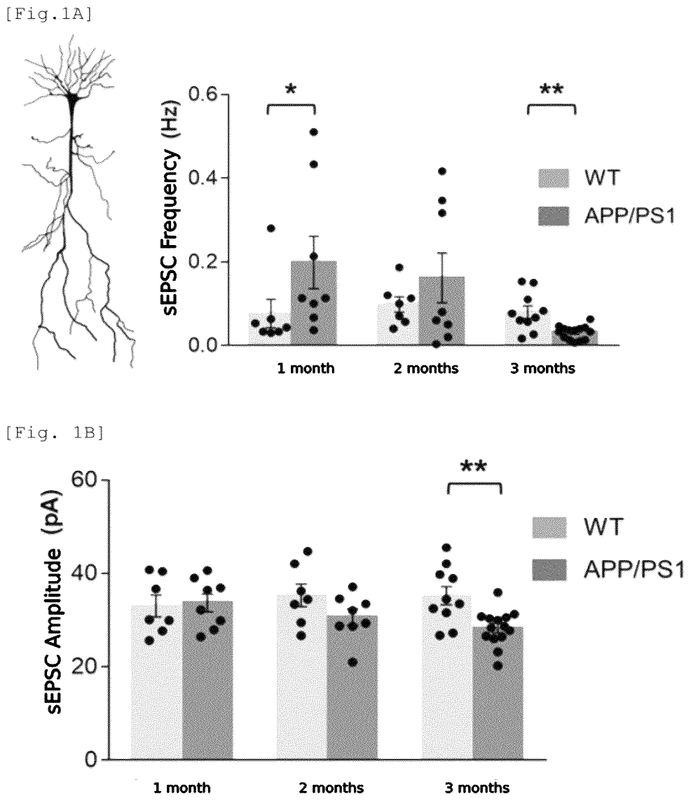

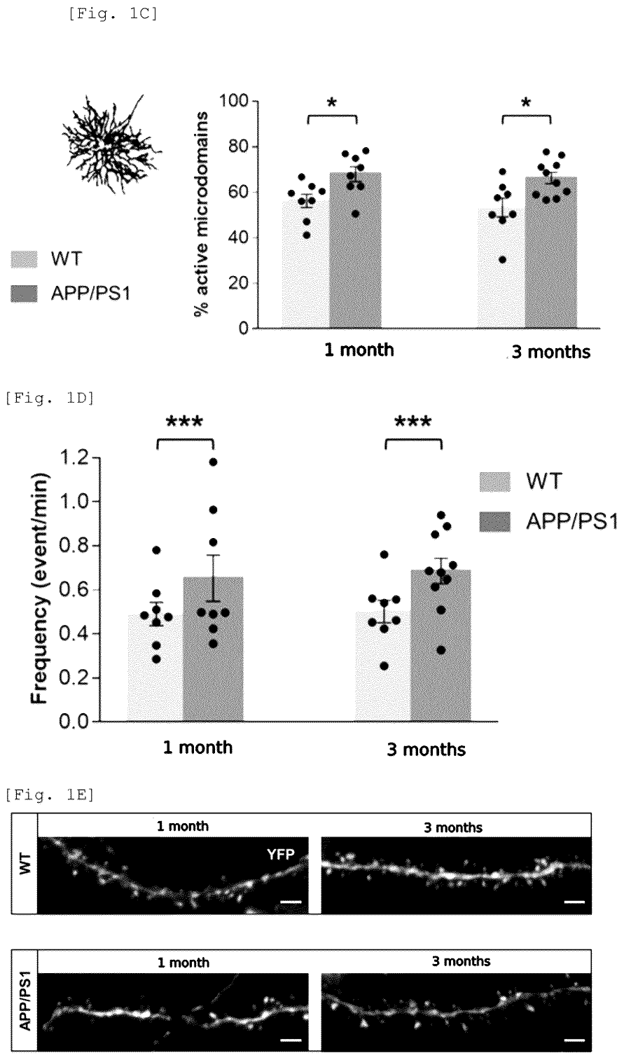

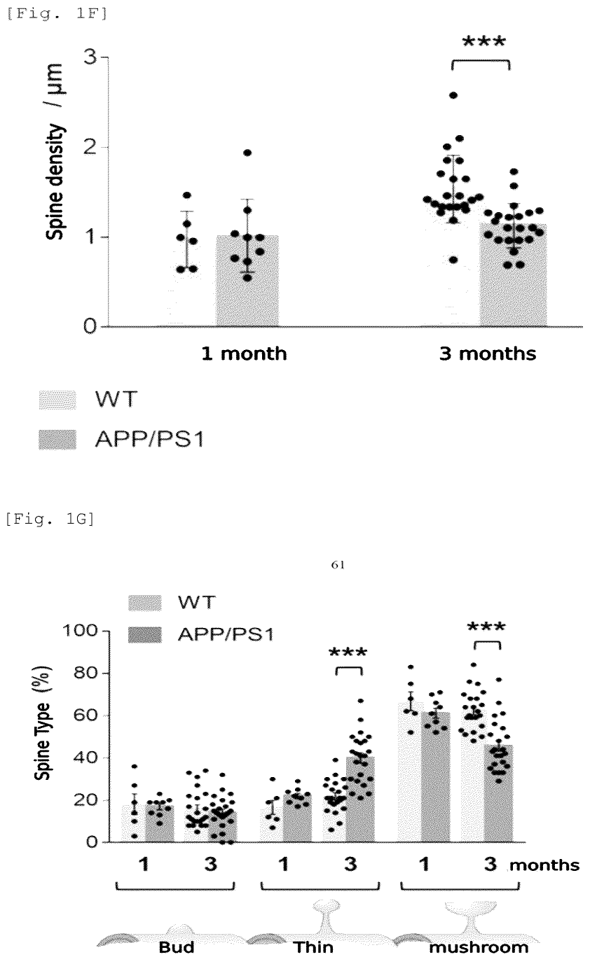

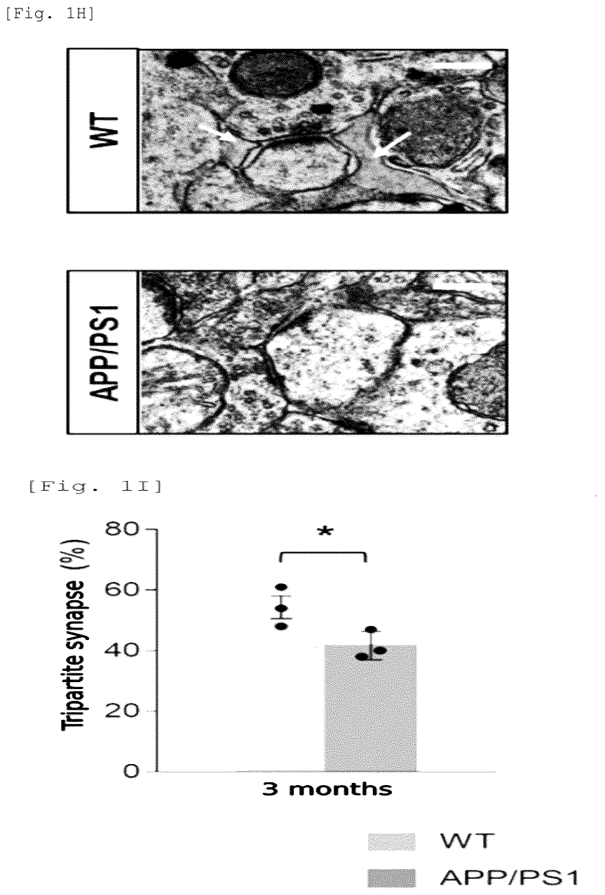

FIG. 1A is a graphical representation of the frequency of sEPSCs (spontaneous excitatory postsynaptic currents) of CA1 pyramidal neurons in WT control (wild-type, light gray) and APP/PS1 transgenic (dark gray) mice of 1-month old (n=8 neurons from 6 APP/PS1 mice and n=7 neurons from 5 WT mice), of 2-months old (n=8 neurons from 5 APP/PS1 mice and n=7 neurons from 4 mice WT) and of 3-months old (n=14 neurons from 7 APP/PS1 mice and n=10 neurons from 5 WT mice), respectively.

FIG. 1B is a graphical representation of the sEPSC amplitude of CA1 pyramidal neurons in WT (light gray) and APP/PS1 (dark gray) mice of 1-month-old (n=8 neurons from 6 APP/PS1 mice and n=7 neurons from 5 WT mice), of 2-months old (n=8 neurons from 5 APP/PS1 mice and n=7 neurons from 4 WT mice) and 3-months old (n=14 neurons from 7 APP/PS1 mice and n=10 neurons from 5 WT mice), respectively.

FIG. 1C is a graphic representation of the activity of microdomains at the level of an astrocyte arborization in WT (light gray) and APP/PS1 (dark gray) mice of 1-month old (n=8 astrocytes from 7 APP/PS1 mice and n=8 astrocytes from 6 WT mice) and of 3-months old (n=10 astrocytes from 4 APP/PS1 mice and n=8 astrocytes from 3 WT mice), respectively.

FIG. 1D is a graphical representation of the frequency of calcium events within each active microdomain in WT (light gray) and APP/PS1 (dark gray) mice (n=650 microdomains from 8 astrocytes in APP/PS1 mice and n=454 microdomains from 8 astrocytes in WT mice) and of 3-months old (n=1504 microdomains from 10 astrocytes in APP/PS1 mice and n=840 microdomains from 8 astrocytes in WT mice). Each point corresponds to the average frequency recorded in each individual astrocyte.

FIG. 1E corresponds to images taken by fluorescence microscopy of portions of CA1 pyramidal neuron dendrites expressing eYFP in Thy1-eYFP-H-APP/PS1-21 transgenic mice and WT mice from the same litter, at 1 month and at 3 months. Scale bar 2 μm.

FIG. 1F is a graphical representation of dendritic spine density per μm in WT (light gray) and APP/PS1 (dark gray) mice of 1-month old (n=32 dendrites from 3 APP/PS1 mice and n=28 dendrites from 3 WT mice) and of 3-months old (n=23 dendrites from 3 APP/PS1 mice and n=22 dendrites from 3 WT mice).

FIG. 1G is a graphical representation of the distribution of the dendritic spine morphology in WT (light gray) and APP/PS1 (dark gray) mice of 1-month old (n=32 dendrites from 3 APP/PS1 mice and n=28 dendrites from 3 WT mice) and of 3-months old (n=23 dendrites from 3 APP/PS1 mice and n=22 dendrites from 3 WT mice).

FIG. 1H corresponds to images taken by electron microscopy of the stratum radiatum in 3-months old WT and APP/PS1 mice showing the presence of synapses enveloped by astrocyte perisynaptic processes in WT mice (white arrows) and the absence of these extensions of astrocytes in APP/PS1 animals. Approximate scale bar: 0.5 μm.

FIG. 1I a graphical representation of the proportion of synapses enveloped by astrocytes in 3-month-old WT (light gray) and APP/PS1 (dark gray) mice (n=480 synapses from 3 APP/PS1 mice and n=441 synapses from 3 WT mice). Each point corresponds to the average proportion measured in each individual mouse.

*p<0.05; **p<0.01 and ***p<0.001 (Mann-Whitney or Fischer test)

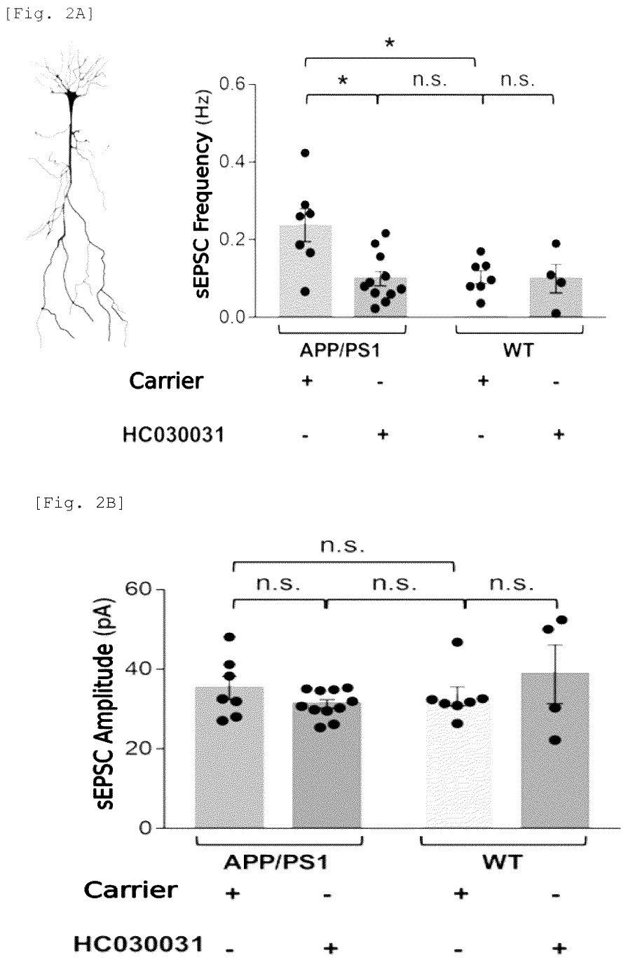

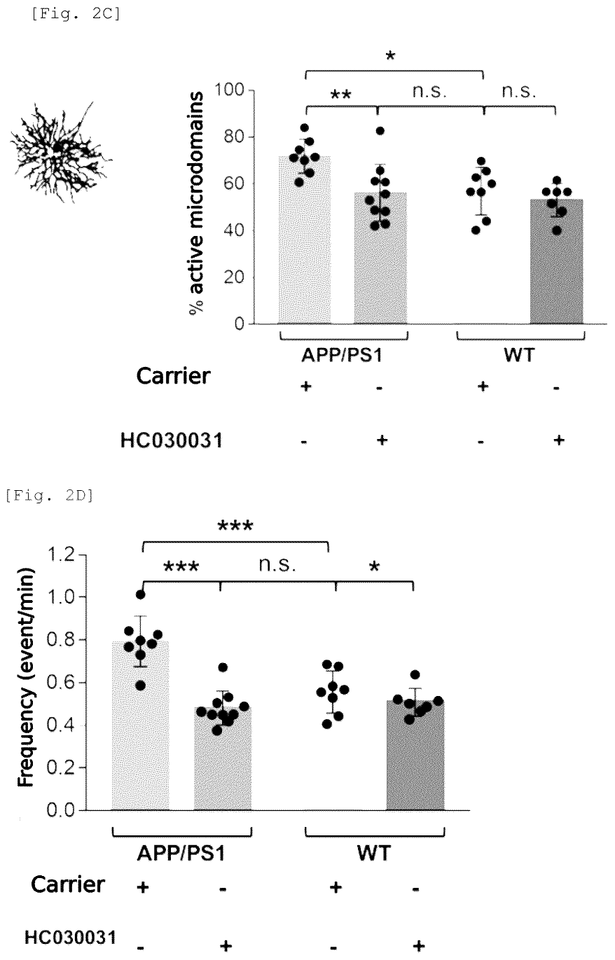

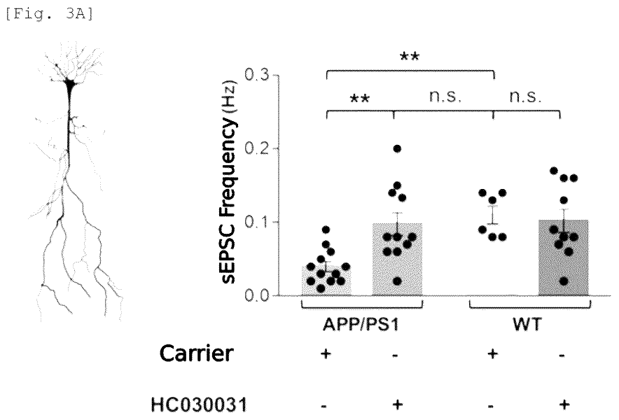

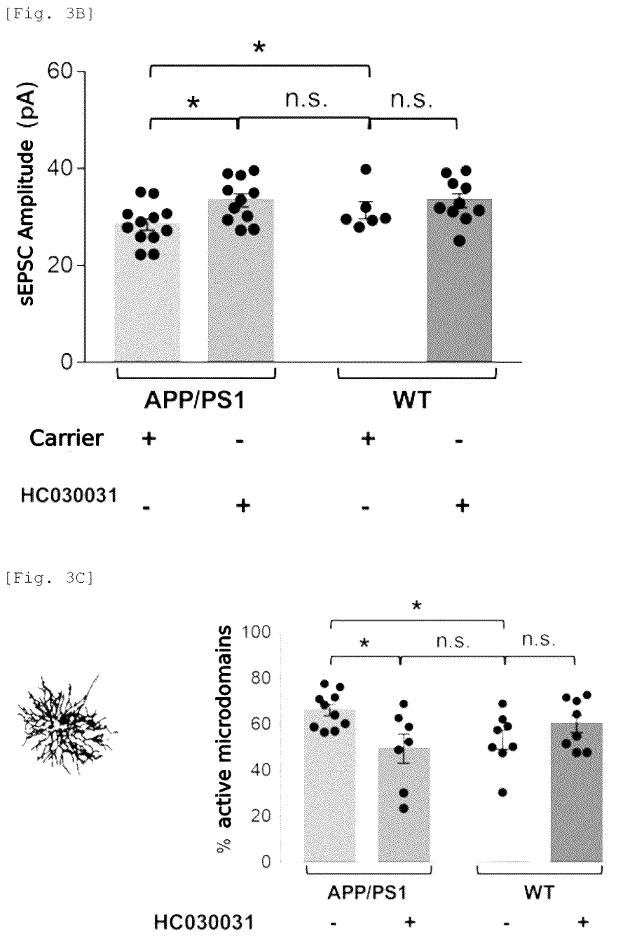

FIG. 2A is a graphical representation of the frequency of sEPSCs (spontaneous excitatory postsynaptic currents) of CA1 pyramidal neurons in 1-month old WT (n=4 neurons from 2 mice treated with HC030031 and n=7 neurons from 4 mice treated with the carrier) and APP/PS1-21 (n=11 neurons from 5 mice treated with HC030031 and n=7 neurons from 5 mice treated with the carrier) mice.

FIG. 2B is a graphical representation of the sEPSC amplitude of CA1 pyramidal neurons in 1-month old WT (n=4 neurons from 2 mice treated with HC030031 and n=7 neurons from 4 mice treated with the carrier) and APP/PS1-21 (n=11 neurons from 5 mice treated with HC030031 and n=7 neurons from 5 mice treated with the carrier) mice.

FIG. 2C is a graphic representation of the calcium activity of the microdomains at the level of an astrocyte arborization in 1-month old WT (n=7 astrocytes from 2 mice treated with HC030031 and n=8 astrocytes from 3 mice treated with theb carrier) and APP/PS1-21 (n=10 astrocytes from 3 mice treated with HC030031 and n=8 astrocytes from 3 mice treated with the carrier) mice.

FIG. 2D is a graphical representation of the frequency of calcium events within each active microdomain in 1-month old WT (n=816 microdomains from 7 astrocytes in HC030031-treated mice and n=995 microdomains from 8 astrocytes in carrier-treated mice) and APP/PS1-21 (n=771 microdomains from 10 astrocytes in HC030031-treated mice and n=1303 microdomains from 8 astrocytes in carrier-treated mice) mice. Each point corresponds to the average frequency recorded in each individual astrocyte.

*p<0.05; **p<0.01 and ***p<0.001 (Kruskal-Wallis test)

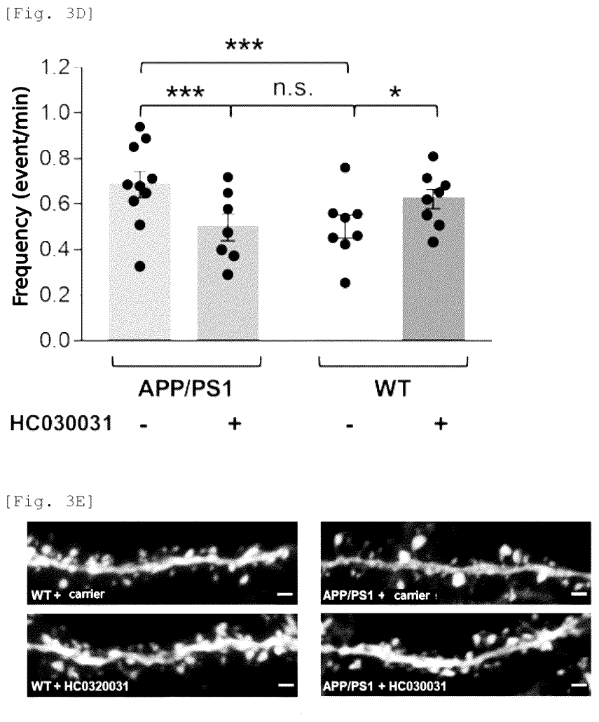

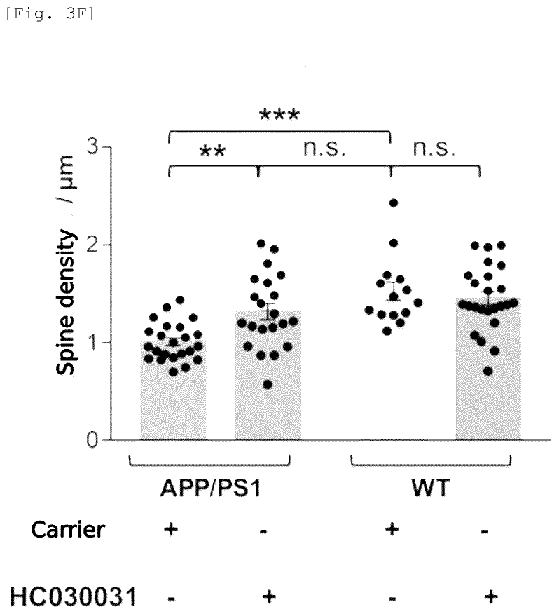

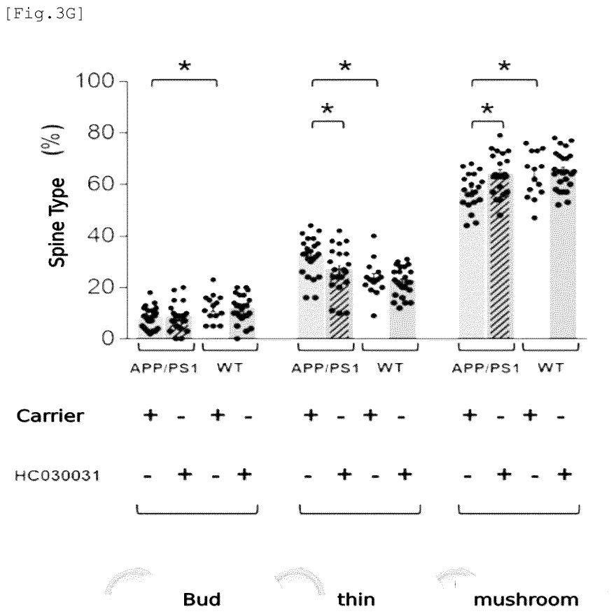

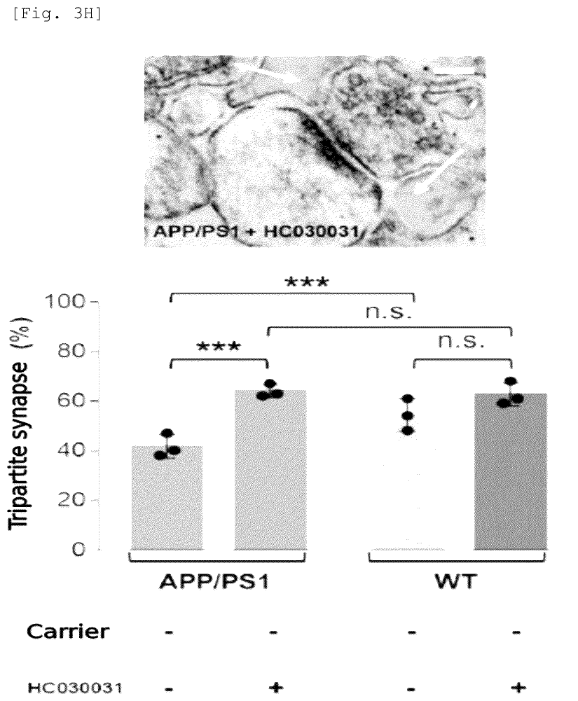

FIG. 3A is a graphical representation of the frequency of sEPSCs of CA1 pyramidal neurons in 3-months old WT (n=11 neurons from 6 mice treated with HC030031 and n=10 neurons from 9 mice treated with the carrier) and APP/PS1-21 (n=11 neurons from 7 mice treated with HC030031 and n=12 neurons from 8 mice treated with the carrier) mice.

FIG. 3B is a graphical representation of the sEPSC amplitude of CA1 pyramidal neurons in 3-month-old WT (n=11 neurons from 6 mice treated with HC030031 and n=10 neurons from 9 mice treated with the carrier) and APP/PS1-21 (n=11 neurons from 7 mice treated with HC030031 and n=12 neurons from 8 mice treated with the carrier) mice.

FIG. 3C is a graphic representation of the calcium activity of the microdomains at the level of an astrocyte arborization in 3-month old WT (n=8 astrocytes from 4 mice treated with HC030031 and n=8 astrocytes from 3 untreated mice) and APP/PS1-21 (n=7 astrocytes from 3 mice treated with HC030031 and n=10 astrocytes from 3 untreated mice) mice.

FIG. 3D is a graphical representation of the frequency of calcium events within astrocyte subregions in 3-months old WT (n=1267 microdomains from 8 astrocytes in mice treated with HC030031 and n=840 microdomains from 8 astrocytes in untreated mice) and APP/PS1-21 (n=1052 microdomains from 7 astrocytes in mice treated with HC030031 and n=1504 microdomains from 10 astrocytes in untreated mice) mice. Each point corresponds to the average frequency recorded in each individual astrocyte.

FIG. 3E corresponds to images taken by fluorescence microscopy of portions of CA1 pyramidal neuron dendrites expressing eYFP in 3-months old mice transgenic Thy1-eYFP-H-APP/PS1-21 and WT mice of the same litter treated with the carrier or with HC030031. Scale bar 2 μm.

FIG. 3F is a graphical representation of dendritic spine density per μm in 3-months old WT (n=24 dendrites from 3 HC030031-treated APP/PS1 mice and n=14 dendrites from 2 carrier-treated mice) and Thy1-eYFP-H-APP/PSl-21 (n=21 dendrites from 3 HC030031-treated APP/PS1 mice and n=14 dendrites from 2 carrier-treated mice) mice.

FIG. 3G is a graphical representation of the distribution of dendritic spine morphology in 3-months old WT and Thy1-eYFP-H-APP/PS1-21 mice.

FIG. 3H is a representation of morphological analysis by electron microscopy of the stratum radiatum in a 3-months old APP/PS1-21 mice treated with HC030031 showing an example of a synapse enveloped by astrocyte perisynaptic processes (arrow). Approximate scale bar: 0.5 μm. The histogram shows the impact of intraperitoneal injection of HC030031 on the proportion of synapses enveloped in 3-months old mice (n=441 synapses from 3 untreated mice and 124 synapses from 3 mice treated with HC030031) and in APP/PS1-21 mice (n=480 synapses from 3 untreated mice and 122 synapses from 3 mice treated with HC030031).

*p<0.05; **p<0.01 and ***p<0.001, n.s. not significant (Kruskal-Wallis or Fischer test)

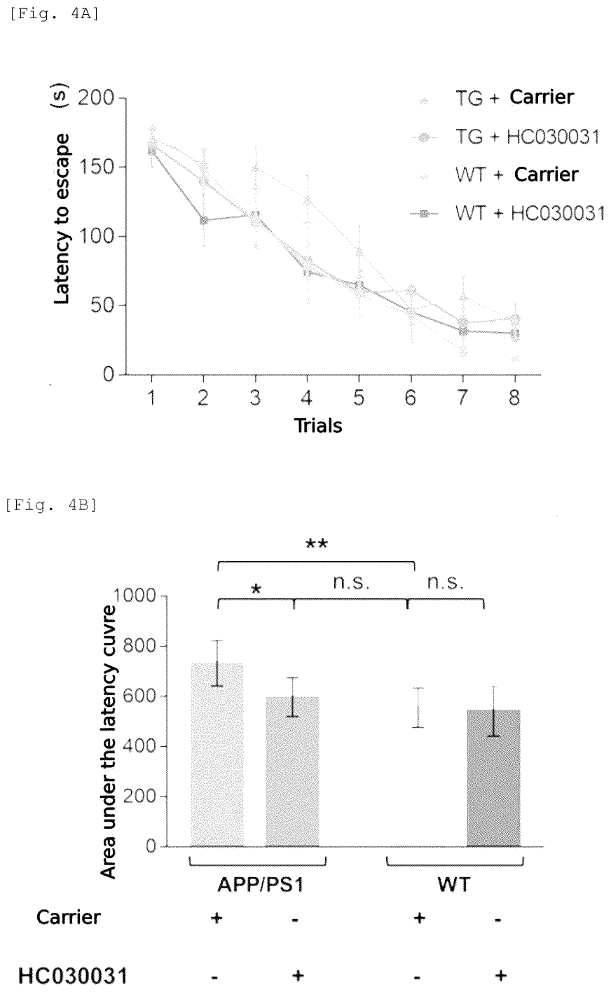

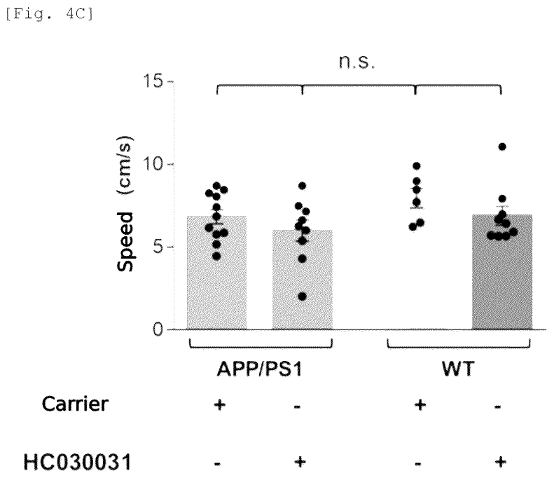

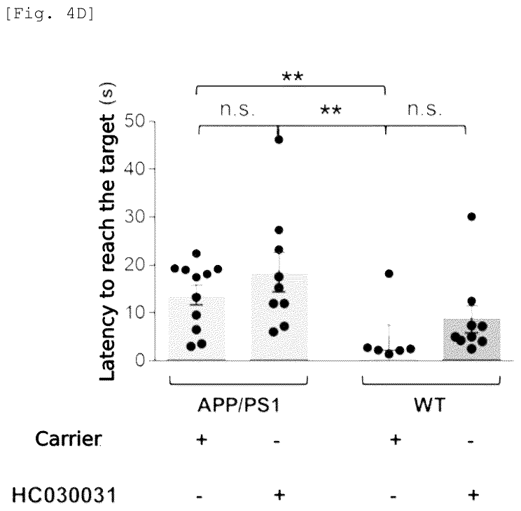

FIG. 4A is a graphical representation of the results of the Barnes maze test to assess spatial task learning in 6-months old WT mice treated with the carrier (n=6 mice), APP/PS1-21 mice treated with the carrier (n=10 mice), WT mice treated with HC030031 (n=9 mice) and APP/PS1-21 mice treated with HC030031 (n=11 mice) daily for 6 months.

FIG. 4B is a graphical representation of the area under the latency curve to reach the Barnes maze escape box in 6-months old mice under the conditions in FIG. 4A.

FIG. 4C is a graphical representation of mouse movement speed through the Barnes maze in 6-months old mice under the conditions in FIG. 4A.

FIG. 4D is a graphical representation of the latency to reach the target during the test phase in 6-months old mice under the conditions in FIG. 4A.

20 *p<0.05; **p<0.01 and ***p<0.001, n.s. not significant (Kruskal-Wallis or 2-factor Anova test)

Referring to FIG. 1A, it can be seen that the frequency of sEPSCs of CA1 neurons in 1-month old APP/PS1-21 mice increases compared to WT control mice. In contrast, at 3 months old, hyperactivity gradually evolved into hypoactivity in APP/PS1-21 mice. Transgenic mice overexpressing mutant APP in combination with mutant PS1 produce high levels of amyloid β and develop amyloid pathology that is similar to that found in the human brain (Radde et al., EMBO Rep., 2006).

In FIG. 1B, it can be noted that the amplitude of sEPSCs is not affected at the age of 1 month but is reduced at the age of 3 months in APP/PS1 mice, which accentuates the marked CA1 neuronal hypoactivity.

In FIG. 1C, the fluorescent calcium probe Fluo-4 made it possible to highlight the active microdomains of the astrocytes. The proportion of active microdomains increased at 1-month old APP/PS1 mice and remained high at 3-months old mice. Similarly, in FIG. 1D, it can be seen that the frequency of calcium events within each active microdomain increased at 1-month old and remained high at 3-months old, revealing stable astrocyte calcium hyperactivity over time.

Thus, the overproduction of Aβ has an early impact on neuronal and astrocyte activity, triggering extended astrocyte hyperactivity in mice aged of 1 month and of 3 months, together with temporary neuronal hyperactivity at 1 month old that evolves into hypoactivity at 3-months old.

Referring to FIG. 1E, captured images show that at 1 month old, there is no difference in dendritic spine density or morphology between control and APP/PS1-21 mice. On the other hand, at 3-months old, it appears that the density decreases in the APP/PS1 mice compared to the control mice of the same age.

These findings could be confirmed by quantification in FIGS. 1F and 1G, where, at 1-month old, there is no difference in the density or morphology of the dendritic spines either in APP/PS1 mice or control mice, whereas at 3-months old, a reduction in the density of dendritic spines is observed and, at the same time, there is an increase in the proportion of thin immature spines and a reduction in the proportion of mature mushroom-shaped spines. The morphology called “bud” corresponds to very immature and non-functional spines while the morphology called “mushroom” corresponds to mature and functional spines.

Electron microscopy imaging presented in FIG. 1H reveals that tripartite synapses (where astrocytes are tightly associated with the synaptic compartment) are predominant in control mice as opposed to APP/PS1 mice.

FIG. 1I effectively illustrates the reduction in the proportion of tripartite synapses in 3-months old APP/PS1 mice compared to control mice from the same litter.

Referring to FIG. 2, it can be seen the effects of chronic treatment with the TRPA1 channel inhibitor (HC030031) on WT control and APP/PS1-21 1-month old mice.

In FIG. 2A, it is observed that treatment with HC030031 prevented the occurrence of hippocampal neuronal hyperactivity in transgenic mice, restoring the frequency of sEPSCs to the physiological value at 1-month old. Treatment with HC030031 had no effect on the control mice of the litter. According to FIG. 2B, the amplitude of sEPSCs was not affected in 1-month old transgenic mice and treatment with HC030031 had no impact on this parameter.

FIGS. 2C and 2D show that treatment with HC030031 prevents the occurrence of hippocampal astrocyte calcium hyperactivity in APP/PS1-21 mice, affecting both the proportions of active microdomains and the frequency of calcium event and these two parameters were restored to the physiological value as represented by the WT+carrier condition.

Referring to FIG. 3, it can be seen the effects of chronic treatment with the TRPA1 channel inhibitor (HC030031) on the control and APP/PS1-21 mice aged of 3-months old.

In FIG. 3A, it is observed that chronic treatment with HC030031 prevented the occurrence of neuronal hypoactivity as evidenced by the restoration of the frequency of sEPSCs to the physiological level obtained in WT mice+carrier. FIG. 3B shows that the amplitude of the sEPSCs has also been restored to its physiological level. Neuronal activity levels in APP/PS1-21 mice treated with HC030031 were similar to those of control mice from the same litter.

As shown in FIGS. 3C and 3D, chronic treatment with HC030031 in APP/PS1-21 mice persisted in reducing astrocyte calcium hyperactivity at 3-months old since both the proportion of active microdomains and the frequency of calcium events within these microdomains were reduced to near physiological level. Chronic treatment with HC030031 had no effect on the proportion of active microdomains in the rest of the litter of control mice and slightly increased the frequency of calcium events within these microdomains.

In FIGS. 3E and 3F, it can be seen that chronic TRPA1 inhibition resulted in the normalization of dendritic spine density to a density observed in control animals.

Finally, in FIG. 3G, it can be seen that the maturation of dendritic spines following chronic treatment with HC030031 on APP/PS1-21 mice approaches that of control animals and that the proportion of thin or mushroom-shaped dendritic spines is restored by chronic treatment. The morphological alterations observed in the APP/PS1-21 mice treated with the carrier are the corollary of the functional alterations described in FIG. 3A (neuronal hypoactivity) and are the prelude to neuronal death. Daily treatment with HC030031 restores these parameters to values close to what is measured in control animals (WT) both in terms of density and synaptic morphology.

As shown in FIG. 3H, along with this protection of dendritic spine integrity, electron microscopic analysis revealed that astrocyte coating defects of hippocampal stratum radiatum synapses are fully restored in transgenic mice treated with HC030031. Furthermore, treatment with HC030031 had no effect on astrocyte coating in control mice.

Referring to FIG. 4, it can be seen the effects of chronic treatment with the TRPA1 channel inhibitor (HC030031) on the memory disorders of 6-months old WT and APP/PS1-21 control mice.

In FIG. 4A shows the learning phase of the test measuring the latency for the mice to escape from the Barnes maze. This test makes it possible to test the spatial working memory of animals. At the end of the learning period of 3 days and 8 trials, the temporal latency until the mouse reaches the escape box decreased over time, both in transgenic mice and WT control mice. However, APP/PS1-21 mice exhibited an increase in the overall time period taken to reach the escape box during this learning phase, which suggests a spatial working memory defect in these transgenic mice. Chronic treatment with HC030031 restores spatial working memory to a level similar to that of WT control mice. FIG. 4B shows the area under the latency curve and characterizes the same trend towards restoration of spatial working memory through chronic treatment with HC030031 of APP/PS1-21 mice.

As shown in FIG. 4C, the motor function, measured by mouse velocity over 3 days, is similar in all tested groups.

In FIG. 4D, the test makes it possible to show the latency to reach the target area. As expected, the latency to find the target area for the first time increased in APP/PS1-21 mice. Therefore, both reference spatial memory and spatial working memory were impaired in 6-months old APP/PS1-21 mice compared to littermate WT control mice. Treatment with HC030031 from the onset of amyloid β peptide overproduction partially restored these memory defects, but the reference memory was not improved unlike the working memory.

Thus, all of these results suggest that early treatment with a TRPA1 channel inhibitor such as HC030031 protects neurons from the toxicity of the Aβ peptide and prevents the occurrence of neuronal dysfunctions characteristic of the early and late pre-symptomatic phases. This functional protection is accompanied by maintenance of the structure of the synapses. These neuronal hyperactivities and then hypoactivities associated with the structural alterations of the synapses are precursors of neuronal death associated with the memory and cognitive symptoms of the pathology.

### EXAMPLES

The following examples illustrate the invention.

Material and Methods

Animals

The used model is a transgenic murine model obtained on a C57BL/6J genetic background which co-expresses the mutated human form of the amyloid precursor protein (mutated APP KM670/671NL) and of presenilin 1 (mutated PS1 L166P) under the control of the neural promoter Thy1. The presence of these two mutations allows increased production of the Aβ peptide from the 14^{th} postnatal day. This leads to cerebral amyloidosis with the appearance of the first senile plaques around 8 postnatal weeks. This amyloid pathology is accompanied by inflammatory gliosis (microgliosis and astrogliosis detectable from 4 months), dendritic spine dystrophy and tau hyperphosphorylation (around 8 months). Cognitive behavioral disorders, and in particular of learning and spatial memory, are developed in around 6 to 8 months. This model was set-up and described by Rebecca Radde in 2006 (Radde et al., EMBO reports, 2006) and is widely used by the scientific community. This model is available in the animal facility of the Institut des Neurosciences de Grenoble and has been bred by the inventors since 2014.

This model being heterozygous, the inventors use mice from the same litters but not expressing the APP/PS1 transgene (wild-type mice, WT) as healthy controls. Both male and female animals are used and the treatment effect is compared based on gender.

The treatment protocol was submitted to the local ethics committee where it received a favorable opinion. It has received authorization from the Ministry of Higher Education and Research for a period of 5 years from March 3^{rd}, 2019 (APAFIS #19142-2018121715504298).

In some experiments, APP/PS1-21 mice were crossed with the Thy1-eYFP-H murine transgenic line which expresses the fluorescent protein eYFP in specific neuron subpopulations.

Treatments

HC030031 [2-(1,3-Dimethyl-2,6-dioxo-1,2,3,6-tetrahydro-7H-purin-7-yl)-N-(4-isopropylphenyl)acetamide] of formula:

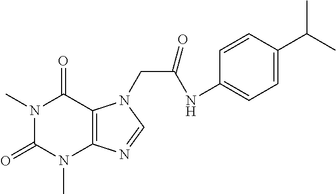 [SMILES: CC(C)c1ccc(NC(=O)Cn2cnc3c2c(=O)n(C)c(=O)n3C)cc1]

is a specific TRPA1 channel inhibitor discovered by Hydra Bioscience in 2007. It is an alkaloid with a xanthine structure that inhibits human and rodent forms of TRPA1 with a CI₅₀ of 6.2 and 7.6 μM respectively (McNamara et al., PNAS, 2007, Eid et al., 2008). The solid form is supplied by Tocris Bioscience (50 mg, ref. #2896) and is dissolved in DMSO at a concentration of 10 mg/ml.

This solution is then diluted to 1 μg/μl in an isotonic solution containing 1% Pluronic F-127 and 0.9% NaCl. The animals are injected daily intraperitoneally at a dose of 5 mg/kg of body mass. Sterile 0.5 ml BD MicroFine syringes with no dead volume, equipped with a 30G (0.3 mm) needle are used for the injections.

The carrier (10% DMSO, 1% Pluronic F-127, 0.9% NaCl) is used as a control and is also injected daily, according to the same procedure, into the WT and APP/PS1 control animals (carrier groups).

The injection is started from the 14^{th} postnatal day (appearance of the expression of the Thy1gene promoter in the hippocampus, triggering the overexpression of Aβ in the APP/PS1-21 murine model)

Preparation of Acute Brain Slices

Coronal sections of the hippocampus (thickness 300 μm) were prepared from APP/PS1-21 transgenic mice (1 month and 3 months old) or from WT mice from the same litter. The mice were sacrificed by decerebration and decapitation. The brain was quickly removed and sectioned in ice-cold ACSF containing (in mM) : 2.5 KCl, 7 MgCl₂, 0.5 CaCl₂, 1.2 NaHPO₄, 25 NaHCO₃, 11D-glucose and 234 sucrose bubbled with 95% of O₂ and 5% of CO₂ with a VT1200S vibratome (Leica, Germany). The sections containing the hippocampus were placed in ACSF containing (in mM) : 126 NaCl, 2.5 KCl, 1.2 MgCl₂, 2.5 CaCl₂, 1.2 NaH₂PO₄ bubbled with 95% of O₂ and 5% of CO₂ and supplemented with 1 mM of sodium pyruvate at room temperature for a recovery period.

Individual Loading of Astrocytes by a Fluorescent Calcium Probe, Fluo-4

300 μm coronal sections were transferred to a chamber allowing constant perfusion with ACSF at room temperature, bubbling with 95% of O₂ and 5% of CO₂ on the upright microscope tray (Eclipse E600 FN, Nikon, France) equipped with a 60× (NA 1.0) water immersion objective and infrared interference contrast optical systems with a CCD camera (Optronis VX45, Germany). 8-11 MSΩ glass pipettes (Harvard Apparatus) were filled with an intracellular solution containing (in mM): 105 K-gluconate, 30 KCl, 10 phosphocreatine, 10 HEPES, 4 ATP-Mg, 0.3 GTP-Tris, 0.2 Fluo-4-pentapotassium salt (Life Technologies), adjusted to pH 7.2 with KOH. The signals were amplified by Axopatch 200B, sampled by a Digidata 1440A interface and recorded by the pClamp8 software (Molecular Devices, USA). Astrocytes were identified on the basis of their morphology, their location in the stratum radiatum and a negative resting potential (between −70 and −80 mV). The membrane potential was maintained at −80 mV. The input resistance was calculated by measuring the current in response to a 10 mV pulse of 80 ms duration near the end of the voltage command. Only passive astrocytes with a linear I/V relationship and a low input resistance (≈50 MΩ are recorded. After the implementation of the whole-cell configuration, the access resistance was constantly monitored and the astrocytes were excluded from this study when this parameter was evolving by more than 20% during the experiment. To allow sufficient diffusion of the fluorescent probe and avoid astrocyte dialysis, the whole-cell setup was limited to less than 5 minutes. Then the patch pipette was gently removed to allow the astrocyte to recover. In order to maximize the diffusion of the probe in the astrocyte extensions, the inventors waited at least 5 minutes before calcium imaging.

Calcium Imaging

Sections loaded with individual astrocytes were subjected to recording in a constantly perfused chamber on the tray of an upright microscope (Eclipse E600, Nikon, France) equipped with a 60× (NA 1.0) water immersion objective and a confocal head (Cl confocal head, Nikon, France). Excitation was performed with 488 nm laser light and the emission was filtered by a 515±15 nm filter. Images were acquired with EZ-Cl software (Nikon, France) at 1.2 s intervals in a single confocal plane over a period of 5 minutes.

Transient calcium events were measured in two-dimensional images over time, in subregions corresponding to the shape of the isolated astrocyte. Regions of interest ROI (≈1 μm²) were placed along the astrocyte extensions located in the focal plane and a ROI was also selected in the soma if it was accessible. Prior to analysis, the raw images were stabilized (if necessary) using ImageJ's Template Matching plugin and filtered with the 3D Hybrid Median Filter. CalSignal software was used to measure the intracellular Ca²⁺ activity, analyze variations in the F fluorescence signal in each ROI. Significant fluorescence changes were detected based on the calculated ΔF/F₀ ratios. FO was calculated for each ROI over the recording period. Based on the ΔF/F₀ ratios, significant fluorescence changes were detected and a calcium event was defined as a significant and continuous increase in signal above a set threshold, followed by a significant and continuous decrease in signal. Thus, ROIs were defined as active when fluorescence increased by at least 2 standard deviations from baseline fluorescence. After peak detection, each transient calcium current was visually checked by the operator.

Electrophysiological Recordings

Whole-cell recordings were performed on visually identified CA1 pyramidal neuron somas. The patch pipettes (4-6 MΩ) were filled with an internal solution containing (in mM): 105 K-gluconate, 30 KCL, 10 phosphocreatine, 10 HEPES, 4 ATP-Mg, 0.3 GTP-Tris, 0.3 EGTA, adjusted to pH 7.2 with KOH.

Spontaneously excitatory postsynaptic currents (sEPSCs) were collected at a membrane holding potential of −60 mV which is close to the GABA reversal potential. All recordings were made at room temperature (22-24° C.) and only a single neuron was studied per section. The sEPSCs and their kinetics were analyzed, after a stabilization period of 10 minutes, 5 minutes from the recordings. Access resistance was constantly monitored and recordings were excluded from this study when this parameter varied more than 20% during the experiment.

Recordings were analyzed using the Clampfit module of pClamp8 software (Molecular Devices, USA) with a single one at 20 pA to exclude miniature sEPSCs.

Immunohistochemistry

Mice were deeply anesthetized by an intraperitoneal injection of 320 mg/kg of sodium pentobarbital and perfused intracardially with 25 mL of 0.1 M PBS, followed by 25 mL of 4% paraformaldehyde in 0.1 M PBS, pH 7.3. Brains were quickly removed, post-fixed overnight at 4° C. in 4% paraformaldehyde, immersed in 20% sucrose in 0.1 M PBS, pH 7.5 overnight, frozen in cooled (−35° C.) isopentane and stored at −30° C. Serial frontal sections (thickness of 30 μm) were cut with a cryostat microtome (HM 500M, Microm, France). Sections were blocked by incubation with 3% of bovine serum albumin in TBS-Tween-Triton (TBSTT) (0.1M Tris base 0.15M NaCl, 0.1% Tween, 0.1% Triton X-100) for 30 minutes (dilution/blocking buffer). Tissue sections were then incubated overnight at 4° C. with either anti-GFAP antibody (Molecular Probes, USA, mouse monoclonal, 1:1000), anti-TRPA1 (Novus, USA, rabbit polyclonal, 1:100) or anti-Iba-1 (Wako, USA, rabbit polyclonal, 1:500). Tissue sections were washed in TBSTT and incubated for 2 hours at room temperature with secondary antibodies conjugated to Cynanine3 (Jackson ImmunoReseach Laboratories, USA, 1:1000) or Alexa 488 (Life Technology, USA, 1:1000). The sections were washed with TBS buffer (0.1M Tris base, 0.15M NaCl), incubated in 1% aqueous Thioflavin S (Sigma, France) for 8 minutes at room temperature, in the dark and washed several times in TBS buffer.

Analysis of Dendritic Spines

Hippocampal sections from Thy1-eYFP-H-APP/PS1-21 mice were imaged using a Zeiss Airyscan module with a 100× (NA 1.4) Plan Apochromat oil immersion objective. Confocal image stacks (increment of 200 nm) were performed with a voxel size of 0.041×0.041×0.2 μm. The 3D analysis of the spine density, of the spine volume and the spine classification was performed using NeuronStudio software (Icahn School of Medicine at Mount Sinai, USA). The length of individual dendrites was automatically measured as well as the number and volume of associated dendritic spines which were classified as bud, thin or mushroom using automated 3D shape classification. For each condition, 10 dendrites were imaged and 700 dendritic spines were analyzed.

Immunoblotting

Dissected hippocampi from APP/PS1-21 mice aged 1 month, 3 months and 6 months were homogenized in cold buffer containing 0.32 M of sucrose and 10 mM of HEPES, pH 7.4. The samples were kept at 4° C. during all stages of the experiments. Homogenates were clarified at 1000 g for 10 minutes to remove nuclei and large debris. The samples in loading buffer were boiled for 10 minutes and equal amounts of protein (20 μg, quantified by micro-BCA assay (Pierce) in duplicate) were loaded onto unlabeled gels precast on 4-20% of Bis-Tris polyacrylamide (Bio-Rad) gradient under denaturing conditions. The proteins were transferred to a polyvinylidene difluoride membrane (Millipore) for 30 minutes at 4° C. The membranes were blocked with 3% of dried milk in Tris-buffered saline (TBS: 10 mM Tris, 150 mM NaCl, pH 7.4) containing 0.1% of Tween for 1 hour at room temperature. The membranes were labeled with an anti-GFAP (Molecular Probes, USA, mouse monoclonal, 1:1000) or anti-TRPA1 (Novus, USA, 1:2000) antibody diluted in 3% dehydrated milk in 0.1% Tween TBS overnight at 4° C. The membranes were washed in 0.2% Tween TBS and labeled with an HRP-conjugated anti-rabbit IgG antibody (Fab′) (Interchim, France, 1:40000) for 45 minutes at room temperature. After the washes, the specific proteins were shown by an ECL detection system (Bio-Rad) with enhanced chemiluminescence and the chemidoc system (Bio-Rad). Chemiluminescence signals were normalized to protein charge signals acquired using unlabeled precast gels (Bio-Rad).

Electron Microscopy

Hippocampi were dissected and fixed with 1.7% of glutaraldehyde in 0.1 M phosphate buffer at pH 7.4 for 48 hours at room temperature. The CA1 region was dissected under a binocular magnifying glass and fixed again for 2 hours in the same solution. Samples were then washed with buffer and post-fixed with 1% of osmium tetroxide and 0.1M phosphate buffer with pH 7.2 for 1 hour at 4° C. After thorough washing with water, the cells were then stained with 1% of uranil acetate with pH 4 in water for 1 hour at 4° C. before being dehydrated by successive alcohol baths (30%-60%-90%-100%-100%-100%) and incubated with a 1:1 epon/ethanol at 100% mixture for 1 hour and several baths of fresh epoxy resin (Sigma-Aldrich, France) for 3 hours. Finally, the samples were included in a capsule filled with resin which was allowed to polymerize for 72 h at 60° C. Ultrathin sections of the samples (60 nm) were cut with an ultramicrotome (Leica, USA). The sections were post-stained with 5% of uranil acetate and 0.4% of lead citrate. Images were acquired with a transmission electron microscope at 80 kV (JEOL 1200EX, Japan), using a digital camera (Veleta, SIS, Olympus, Germany), the magnification was set at 50,000×. The presence of an astrocyte extension in contact with the synapse was estimated in the stratum radiatum from 42 randomly selected sections, from three mice per condition. Astrocyte processes were identified by their relatively clear cytoplasm, their angular shape compared to their smoother neuronal counterparts, and by the presence of glycogen granules.

Barnes Maze Experiments

The mice were placed in the center of a well-lit open platform (BioSeb, diameter 120 cm). The platform includes 20 holes (diameter 5 cm) with an escape box (target) attached below any of the holes. On the first day of training, the mice were first placed in the middle of the platform and gently guided to the target after 5 minutes of free exploration. Once the mice entered the escape box, they remained there for 1 minute before being returned to their cage. During the 3 days of the training/acquisition phase, the mice performed 3 trials per day with an interval between trials of 20 minutes. The mice had 180 seconds to find the escape box or were gently guided thereto. On day 4 (test day), the escape box was removed and the mice were allowed to freely explore the maze for 60 seconds. The time taken to reach the target (latency) and the exploration time of the target area (target hole±1) were analyzed. All data were recorded and analyzed with EthoVision XT9 (Noldus).

Statistical Analysis

The sample size for the different data sets is mentioned in the description of the Figures. Results are expressed as mean±standard deviation from independent biological samples accompanied by the distribution of experimental points. Data were analyzed using GraphPad Prism 6.0 software. Comparisons between the two groups were made using the two-variable Mann-Whitney test. Kruskal-Wallis test followed by Dunn's multiple comparison test was used for multiple comparisons. The proportions of tripartite synapses were compared with Fischer's exact test. The inventors checked for possible gender dependence of neuronal activity, astrocyte activity and memory performance in the treated groups and found no significant difference, so the results were pooled for analysis. The levels of significance are as follows: *p<0.05, 5 **p<0.01 and ***p<0.001, and n.s. not significant.

Results:

Structural and Functional Alterations of Neurons and Astrocytes in the Early and Intermediate Stages of Alzheimer's Disease

The inventors studied the progression of the activity of neurons and astrocytes in the hippocampus of APP/PS1-21 mice. Transgenic mice overexpressing mutant APP in combination with mutant PS1 produce high levels of amyloid β and develop an amyloid pathology that is similar to that found in the human brain. Significant levels of Aβ are detected from the age of 1 month in this model, while the first amyloid plaques appear in the hippocampus at around the age of 3 to 4 months. These ages may be associated with the early and intermediate stages of Alzheimer's disease progression.

The inventors recorded spontaneous excitatory postsynaptic currents (sEPSCs) by performing whole-cell patch-clamp recordings on CA1 neuron cell bodies in 1-, 2- and 3-month(s) old mice. The results showed that the frequency of sEPSCs of CA1 neurons in 1-month old APP/PS1-21 mice (0.20±0.06 Hz in APP/PS1 mice vs. 0.08±0.03 Hz in WT mice; p=0.0266—FIG. 1A) increases. This hyperactivity gradually evolved into a hypoactivity in 3-months old APP/PS1-21 mice (0.03±0.01 Hz in APP/PS1 mice vs. 0.08±0.02 Hz in WT mice; p=0.0029 -FIG. 1A). The amplitude of sEPSCs was not affected at 1 month old (33.7±1.9 pA in APP/PS1 mice vs. 33.0±2.3 pA in WT mice; p=0.9551—FIG. 1B) and was reduced at the age of 3 months (28.9±1.0 pA in APP/PS1 mice vs. 35.2±1.9 pA in WT mice; p=0.0038—FIG. 1B), which accentuated the marked CA1 neuronal hypoactivity.

To study the astrocyte calcium activity, the inventors loaded individual astrocytes with a fluorescent calcium probe Fluo-4 allowing access to the activity of the microdomains in the whole of the astrocyte territory and in particular at the level of the cellular extensions. The proportion of active microdomains increased in 1-month old APP/PS1 mice (67.9±3.3% in APP/PS1 mice vs. 56.2±2.9% in WT mice; p=0.0145—FIG. 1C) and remained high in 3-months old mice (66.2±2.5% in APP/PS1 mice vs. 53.3±4.1% in WT mice; p=0.0266—FIG. 1C). The frequency of calcium events within each active microdomain increased at age of 1 month (0.65±0.10 event/minute in APP/PS1 mice vs. 0.49±0.05 event/minute in WT mice; p<0.001—FIG. 1D) and remained high at age of 3 months (0.68±0.06 event/minute in APP/PS1 mice vs. 0.50±0.05 event/minute in WT mice p<0.001—FIG. 1D, revealing a stable astrocyte calcium hyperactivity over time. Thus, the overproduction of Aβ has an early impact on neuronal and astrocyte activity, triggering an extended astrocyte hyperactivity in mice aged 1 and 3 month(s), together with a temporary neuronal hyperactivity at 1-month old that progresses to hypoactivity at 3-months old.

At the structural level, the inventors analyzed the density of dendritic spines and their morphology in pyramidal CA1 neurons in Thy1-eYFP-H transgenic mice crossed with APP/PS1-21 mice (FIG. 1E). At 1-month old, there was no difference in the density or morphology of the dendritic spines either in the APP/PS1 mice or the WT mice (FIG. 1F, FIG. 1G), whereas at 3-months old, a reduction in the density of dendritic spines is observed (1.13±0.05 spine/μm in APP/PS1 mice vs. 1.54±0.08 spine/μm in WT mice; p<0.001—FIG. 1F). At the same time, there is an increase in the proportion of immature thin spine (40±2.5% in APP/PS1 mice vs. 21.9±1.6 in WT mice; p<0.001—FIG. 1G) and a reduction in the proportion of mature mushroom-shaped spine (45.7±2.5% in APP/PS1 mice vs. 62.4±2.0% in WT mice; p<0.001—FIG. 1G). This spinal dystrophy is accompanied by a loss of astrocyte coating of the synapses of the stratum radiatum (FIG. 1H). In 3-months old transgenic mice, a reduction in the proportion of tripartite synapses is observed (41.7±2.8% of tripartite synapses in APP/PS1 mice vs. 54.3±3.7 in WT mice; p<0.0001—FIG. 1I). Thus, the establishment of the reduction in the frequency and amplitude of CA1 neuronal sEPSCs was concomitant with the significant reduction in the density and maturity of the dendritic spines at the age of 3 months in the APP/PS1 mice, together with a reduction of astrocyte coating of synapses.

The following examples illustrate the present invention without however limiting its scope.

### Example 1

### Chronic TRPA1 Inhibition Completely Restores CA1 Astrocyte Hyperactivity and Neuronal Hyperactivity in 1-Month Old APP/PS1-21 Mice

The inventors have shown that astrocytes contribute to the toxicity of the amyloid β (Aβ) peptide and are associated with the establishment of the early neuronal hyperactivity via the involvement of TRPA1 calcium channels.

On a model of acute mouse brain slices, it was demonstrated that a rapid and generalized calcium hyperactivity induced by Aβ occurs in the arborization of astrocytes of the hippocampus (stratum radiatum) long before the implementation of inflammatory processes (APP/PS1-21 mice, 1-month old). This astrocyte hyperactivity is fully restored by acute blocking of the TRPA1 channel by HC030031.

The inventors have observed that this TRPA1 channel-dependent hyperactivity influences neighboring neurons, triggering an increase in neuronal activity in the CA1 region of the hippocampus. This neuronal hyperactivity has recently been identified as being a characteristic of the prodromal phase of Alzheimer's disease and associated with precursor memory deficits.

Similarly to astrocyte hyperactivity, this neuronal hyperactivity is fully restored by acute in vitro application of HC030031 in 1-month old APP/PS1-21 transgenic animals. The results led to the hypothesis that an early inhibition of the TRPA1 channel could prevent the establishment of neuronal hyperactivity and this was tested in vivo.

The specific TRPA1 inhibitor was administered daily intraperitoneally (5 mg/kg of body mass) from the 14^{th} postnatal day up to one month in APP/PS1-21 mice. The same volume of carrier was administered as a control in groups of age- and sex-matched control mice. The inventors observed that the pharmacological treatment with HC030031 made it possible to prevent the occurrence of astrocyte calcium hyperactivity of the hippocampus in APP/PS1-21 mice, affecting both the proportions of active microdomains (56.1±3.9% in APP/PS1-21 mice vs. 71.8±2.5% in control mice; p=0.0439—FIG. 2C) and the calcium event frequency (0.48±0.02 calcium event/minute in HC030031-treated mice vs. 0.79±0.04 calcium event/minute in carrier-treated control mice; p<0.001—FIG. 2D). These two parameters were restored to values close to the physiological value (56.9±3.6% of active microdomains and 0.56±0.03% calcium event/minute in carrier-treated WT mice; p=0.7211 and 0.1456, respectively).

Conversely, HC030031 had no effect on the proportion of active microdomains in the litter of WT control mice (53.0±2.7% in HC030031-treated mice vs. 56.9±3.6% in carrier-treated mice, p=0.8627—FIG. 2C) and slightly decreased the frequency of calcium events within the microdomains (0.51±0.02 calcium event/min in HC030031-treated mice vs. 0.56±0.03 calcium event/minute in carrier-treated mice; p=0.031 -FIG. 2D).

Concomitantly, the treatment with HC030031 prevented the occurrence of hippocampal neuronal hyperactivity in transgenic mice (0.10±0.02 Hz in HC030031-treated mice vs. 0.24±0.04 Hz in carrier-treated mice; p=0.0311—FIG. 2A), restoring the frequency of sEPSCs to values near to the physiological value (0.10±0.02 Hz in the carrier-treated WT mice; p=0.7213) at 1 month. The treatment with HC030031 had no effect on mice from the WT litter (0.10±0.04 Hz with the treatment with HC030031 vs. 0.10±0.02 Hz with the treatment with carrier; p>0.999—FIG. 2A). The sEPSC amplitude was not affected in 1-month old transgenic mice (FIG. 2B) and treatment with HC030031 had no impact on this parameter p>0.999; FIG. 2B).

These data suggest that inhibition of TRPA1 from the establishment of Aβ overproduction in the transgenic mice model of Alzheimer's disease was sufficient to prevent hippocampal neuronal hyperactivity at 1 month, confirming that astrocytes play a key role in this early β-amyloid peptide-dependent toxicity. Under these conditions, the neuronal activity of animals at 1 month is completely restored to basal values identical to those of healthy animals. Thus, the early inhibition of TRPA1 by HC030031 prevents the occurrence of neuronal hyperactivity in these APP/PS1-21 mice, suggesting a neuroprotective role.

### Example 2

### Chronic TRPA1 Inhibition Completely Restores CA1 Neuronal Hypoactivity, Astrocyte Calcium Hyperactivity and Morphological Alteration of Dendritic Spines in 3-Months Old APP/PS1-21 Mice

In this transgenic model at 3 months, the inventors observed the establishment of several concomitant phenomena in the CA1 area of the hippocampus: a reduction in the density of the dendritic spines (synapses) of the CA1 neurons, a reduction in the astrocyte coating of the remaining synapses, a reduction in neuronal activity (hypoactivity), a persistence of astrocyte hyperactivity and the progressive appearance of the first senile plaques. These phenomena precede the memory and cognitive behavioral disorders that set at around 6 months in these transgenic mice. The inventors have shown that the daily injection of HC030031 from the 14^{th} to the 90^{th} postnatal day prevents the establishment of neuronal hypoactivity and restores both the frequency and the amplitude of the spontaneous post-synaptic currents in the CA1 region of the hippocampus. This functional protection is accompanied by a protection of the structural integrity of the synapse (density, morphology and astrocyte coating of the dendritic spines). Very interestingly, the injection of HC030031 has no effect on the WT control animals, neither at the functional level nor at the structural level.

Neuronal hyperactivity is presumed to be a key element in the early stages of Alzheimer's disease, triggering a vicious cycle leading to dysregulation of the entire network. The inventors wondered whether blocking Aβ-induced TRPA1 activation could protect the neurons from this vicious circle.

The inventors administered the TRPA1 channel specific inhibitor (HC030031) or the carrier intraperitoneally (5 mg/kg of body mass) from the 14^{th} postnatal day up to 3 months of APP/PS1-21 mice. These experiments revealed that chronic inhibition of TRPA1 in APP-PS1-21 mice prevents the occurrence of neuronal hypoactivity, whether in the frequency of sEPSCs (0.10±0.01 Hz for the treatment with HC030031 vs. 0.04±0.01 for the carrier; p=0.01—FIG. 3A) or in the amplitude (33.4±1.4 pA for the treatment with HC030031 vs. 28.5±1.2 for the carrier; p=0.0358—FIG. 3B). Neuronal activity levels in APP/PS1-21 mice treated with HC030031 were similar to those of littermate WT mice (0.11±0.01 Hz and 32.2±1.1 pA in WT mice treated with the carrier; p=0.386 and 0.6362, respectively). This chronic treatment had no effect on the frequency of the sEPSCs or the amplitude in WT mice (p>0.999 for both; FIG. 3A-FIG. 3B).

In parallel, chronic treatment with HC030031 in APP/PS1-21 mice persisted in reducing astrocyte calcium hyperactivity at 3 months since both the proportion of active microdomains (49.4±6.4% in mice treated with HC030031 vs. 66.2±2.5% in untreated mice; p=0.033—FIG. 3C) and the frequency of calcium events within these microdomains (0.50±0.06 event/minute in treated mice vs. 0.68±0.06 event/minute in untreated mice; p<0.0001—FIG. 3D) were reduced to a physiological level. The chronic treatment with HC030031 had no effect on the proportion of active microdomains in the rest of the WT mice litter (60.2±3.8% in HC030031-treated mice vs. 53.2±4.1% in untreated mice; p=0.2786—FIG. 3E) and slightly increased the frequency of calcium events within these microdomains (0.62±0.04 calcium event/minute in mice treated with HC030031 vs. 0.50±0.05 in untreated mice; p=0.001—FIG. 3F).

To assess the impact of chronic treatment on the density and morphology of dendritic spines, the inventors administered the TRPA1 inhibitor or the carrier from the 14^{th} postnatal day up to 3 months in Thy1-eYFP-H-APP/PS1-21 transgenic mice. The chronic inhibition of TRPA1 resulted in normalization of the dendritic spine density (1.32±0.08 spine/μm in HC030031-treated mice vs. 1.01±0.04 spine/μm in carrier-treated mice; p=0.0019—FIG. 3F) and their maturation (26.7±2.0% thin spines in HC030031-treated mice vs. 32.0±1.6% in carrier-treated mice and 64.0±1.7% mushroom-shaped spines in HC030031-treated mice vs. 57.3±1.5% in carrier-treated mice; p=0.05 and 0.0117, respectively—FIG. 3G).

Along with this protection of dendritic spine structural integrity, an electron microscopic analysis revealed that astrocyte coverage of stratum radiatum synapses was fully restored in HC030031-treated transgenic mice (64.0±1.5% of synapses enveloped in APP/PS1 mice treated with HC030031 vs 41.7±2.7% in untreated APP/PS1 mice; p<0.0001—FIG. 3H). Furthermore, The treatment with HC030031 had no effect on astrocyte coating in WT mice (62.7±2.7% of synapses enveloped in WT mice treated with HC030031 vs 54.3±3.7% in untreated WT mice; p=0.08—FIG. 3H).

Generally, these data showed that early chronic inhibition of the TRPA1 channel prevents the appearance of neuronal hyperactivity which seems to be the driving force behind early progressive deficiencies triggering loss of functional dendritic spines and neuronal hypoactivity.

### Example 3

### Chronic TRPA1 Inhibition Prevents Working Memory Defects in 6-Months Old APP/PS1-21 Mice

The working memory is perhaps one of the most studied aspects of memory impairment in Alzheimer's disease. The inventors administered daily the TRPA1 channel specific inhibitor (HC030031) or the carrier intraperitoneally (5 mg/kg of body mass) from the 14^{th} postnatal day up to 6 months in APP/PS1-21 and wild-type WT mice.

A hippocampus-dependent spatial reference and working memory task experiment was performed using the Barnes maze paradigm test in 6-months old mice. During the 3-day acquisition period (8 trials), the temporal latency until the mouse reaches the escape box decreased over time in both transgenic mice and WT control mice. This indicates that all mice learned to use spatial indications to find the escape box (FIG. 4A). However, APP/PS1-21 mice exhibited an increase in the time period required to reach the escape box during this learning phase, thus suggesting a spatial working memory defect in these transgenic mice (evidenced by area under the curve for latency: 731.8±91.1 in carrier-treated APP/PS1-21 mice vs. 554.1±78.9 in carrier-treated WT mice; p=0.0012—FIG. 4B).

The motor function, measured by mice velocity over 3 days, was similar in all groups (p=0.1718—FIG. 4C). To assess reference memory, 24 hours after the last day of acquisition, mice were subjected to a trial in which the escape box was removed. As expected, the latency to find the target area for the first time increased in APP/PS1-21 mice (13.74±2.1 s in carrier-treated transgenic mice vs. 4.8±2.7 s in carrier-treated WT mice; p=0.0057—FIG. 4D). Therefore, both reference spatial memory and spatial working memory were impaired in 6-months old APP/PS1-21 mice compared to littermate WT control mice. The treatment with HC030031 from the onset of amyloid β peptide overproduction partially restored these memory defects. Indeed, the working memory defects highlighted during the learning phase in the transgenic mice were completely restored (AUC: 595.9±77.2 in HC030031-treated APP/PS1-21 mice vs. 731.8±91.1 in carrier-treated APP/PS1-21 mice; p=0.0089—FIG. 4A and FIG. 4B) and became similar to WT mice (554.1±78.9; p=0.8054). Conversely, long-term treatment with HC030031 did not improve reference memory since the first latency to the target area during the trial was similarly increased in transgenic mice treated with HC030031 (18.5±4.2 s in APP/PS1-21 mice treated with HC030031 vs. 13.74±2.1 s in carrier-treated APP/PS1-21 mice; p=0.6109—FIG. 4D). The chronic treatment with HC030031 had no impact on reference and working memory performance in WT control mice (FIG. 4A-FIG. 4D).

The results presented hereinabove show that a prophylactic treatment with a TRPA1 channel inhibitor normalizes the astrocyte activity to levels normally detected in wild-type animals. This normalization prevents the implementation of hippocampal neuronal hyperactivity, which confirms the pivotal role of astrocytes in preserving synapse integrity. Blocking the TRPA1 channel therefore appears sufficient to prevent the vicious circle of neurodegeneration that is encountered in Alzheimer's disease.

In addition, the inventors observed that, in the absence of treatment, neuronal hypoactivity and astrocyte hyperactivity were no longer dependent on the activation of the TRPA1 channel in 3-months old transgenic mice. This astrocyte functional remodeling is the corollary of morphological remodeling and the establishment of an inflammatory phenotype within these cells. The role of astrocytes in an advanced stage of Alzheimer's disease is well known since the astrocyte inflammatory phenotype and astrogliosis have long been associated with the formation of plaques, potentially containing the toxic effect of amyloid β species in the compaction of plaques but inducing a deleterious neuroinflammation.

In an aged murine model of Alzheimer's disease, astrocyte calcium activity has been shown to dramatically increase and become synchronous with nearby cortical Aβ plaques (Kuchibhotla, K. V. et al., Science 2009 Feb. 27; 323(5918); Delekate, A. et al., Nat. Commun. 5, 5422 (2014)). Interestingly, this astrocyte hyperactivity involved astrocyte P2YR in both cortical and hippocampal areas, suggesting that the astrocyte calcium signaling reorganized in late pathogenic stages involving a purinergic pathway. Indeed, inflammatory reactive astrocytes are accompanied by structural changes that likely affect the molecular signaling machinery within astrocyte microdomains. It may therefore be that, in later stages, the involvement of TRPA1 points to the regulation of Aβ-mediated inflammatory response in astrocytes since its genetic ablation attenuates astrocyte-derived inflammatory processes in late stages of the Alzheimer's disease.

Therefore, it is possible that inhibition of the TRPA1 channel can block the progression of Alzheimer's disease through several routes, one is neuroprotective, the other is anti-inflammatory.

The present invention thus suggests the use of a TRPA1 channel inhibitor for the prevention and the treatment of Alzheimer's disease, in particular from an early, asymptomatic stage. The inhibitor according to the invention has a role both as a neuroprotective agent and as an anti-inflammatory agent to prevent the neurodegenerative and inflammatory mechanisms involved in Alzheimer's disease.

1. A TRPA1 calcium channel inhibitor for use as a neuroprotectant in the treatment and/or prevention of the early stages of Alzheimer's disease and as an anti-inflammatory in the treatment and/or prevention of the neuroinflammatory processes in Alzheimer's disease.

2. The TRPA1 calcium channel inhibitor for use according to claim 1, wherein the inhibitor is selected from the group consisting of HC030031, Chembridge-5861528, A-967079, AP-18, GRC-17536, CB-625, ODM-108, GSK205, GDC-0334 and their derivatives.

3. The TRPA1 calcium channel inhibitor for use according to claim 1, wherein the inhibitor is HC030331.

4. The TRPA1 calcium channel inhibitor for the use according to claim 1, intended to be administered to a subject in need thereof from the prodromal phase of Alzheimer's disease characterized by neuronal hyperactivity in the hippocampus detectable by brain imaging and mild memory disorders.

5. The TRPA1 calcium channel inhibitor for use according to claim 1, for administration to a subject in need thereof in a pharmaceutically effective amount.

6. The TRPA1 calcium channel inhibitor for use according to claim 1, wherein the inhibitor is to be administered by any one of the routes of administration selected from the group consisting of the oral route, the intravenous route, the intra-arterial route, the intradermal route, the intraperitoneal route, the intracardiac route, the intracerebroventricular route, the transdermal route, the topical route, the subcutaneous route, the nasal route or the pulmonary route.

7. A pharmaceutical composition for use in the treatment and/or prevention of the early stages of Alzheimer's disease and in the treatment and/or prevention of the neuroinflammatory processes of Alzheimer's disease, comprising:
at least the inhibitor according to claim 1;
at least one pharmaceutically acceptable excipient.

8. The pharmaceutical composition for use in the treatment and/or prevention of the early stages of Alzheimer's disease and in the treatment and/or prevention of the neuroinflammatory processes of Alzheimer's disease according to claim 7, wherein the composition is intended to be administered to a subject in need thereof in a pharmaceutically effective amount.

9. The pharmaceutical composition for use in the treatment and/or prevention of the early stages of Alzheimer's disease and in the treatment and/or prevention of neuroinflammatory processes of Alzheimer's disease according to claim 7, wherein the composition is intended to be administered to a subject in need thereof by any one of the routes of administration selected from the group consisting of the oral route, the intravenous route, the intra-arterial route, the intradermal route, the intraperitoneal route, the intracardiac route, the intracerebroventricular route, the transdermal route, the topical route, the subcutaneous route, the nasal route or the pulmonary route.

10. The pharmaceutical composition for use in the treatment and/or prevention of the early stages of Alzheimer's disease and in the treatment and/or prevention of neuroinflammatory processes of Alzheimer's disease according to claim 5, wherein the composition is intended to be administered to a subject in need thereof from the prodromal phase of Alzheimer's disease characterized by neuronal hyperactivity in the hippocampus detectable by brain imaging and mild memory disorders.
